SEQUENCE LISTING (14 sequences)

SEQ ID NO: 1 - protein, 178 aa, Artificial Sequence
  Protein translated from synthetic DNA
ADSSPLLQFGGQVRQRYLYTDDAQRTEAHLEIREDGTVGGAADQSPESLLQLKALKPGVIQILGVRTPRFLCQRPDGALYGSLHFDPEACSFRELLLEDGYNVYQSEAHGLPLHLPGNKSPHRDPAPRGPARFLPLPFLPPALPEPPGILGPQPPDVGSSDPLSMVGPSQGRSPSYAS

SEQ ID NO: 2 - protein, 784 aa, Artificial Sequence
  Protein translated from synthetic DNA
ADSSPLLQFGGQVRQRYLYTDDAQRTEAHLEIREDGTVGGAADQSPESLLQLKALKPGVIQILGVRTPRFLCQRPDGALYGSLHFDPEACSFRELLLEDGYNVYQSEAHGLPLHLPGNKSPHRDPAPRGPARFLPLPFLPPALPEPPGILGPQPPDVGSSDPLSMVGPSQGRSPSYASGGGGSGGGGSGGGGSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 3 - protein, 784 aa, Artificial Sequence
  Protein translated from synthetic DNA
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSADSSPLLQFGGQVRQRYLYTDDAQRTEAHLEIREDGTVGGAADQSPESLLQLKALKPGVIQILGVRTPRFLCQRPDGALYGSLHFDPEACSFRELLLEDGYNVYQSEAHGLPLHLPGNKSPHRDPAPRGPARFLPLPFLPPALPEPPGILGPQPPDVGSSDPLSMVGPSQGRSPSYAS

SEQ ID NO: 4 - protein, 398 aa, Artificial Sequence
  Protein translated from synthetic DNA
ADSSPLLQFGGQVRQRYLYTDDAQRTEAHLEIREDGTVGGAADQSPESLLQLKALKPGVIQILGVRTPRFLCQRPDGALYGSLHFDPEACSFRELLLEDGYNVYQSEAHGLPLHLPGNKSPHRDPAPRGPARFLPLPFLPPALPEPPGILGPQPPDVGSSDPLSMVGPSQGRSPSYASGGGGSGGGGSGGGGSVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - protein, 398 aa, Artificial Sequence
  Protein translated from synthetic DNA
VEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSADSSPLLQFGGQVRQRYLYTDDAQRTEAHLEIREDGTVGGAADQSPESLLQLKALKPGVIQILGVRTPRFLCQRPDGALYGSLHFDPEACSFRELLLEDGYNVYQSEAHGLPLHLPGNKSPHRDPAPRGPARFLPLPFLPPALPEPPGILGPQPPDVGSSDPLSMVGPSQGRSPSYAS

SEQ ID NO: 6 - DNA, 537 bp, Artificial Sequence
  Synthetic DNA
gcagactccagtcctctcctgcaattcgggggccaagtccggcagcggtacctctacacagatgatgcccagcgtacagaagcccacctggagatcagggaggatgggacggtggggggcgctgctgaccagagccccgaaagtctcctgcagctgaaagccttgaagccgggagttattcaaatcttgggagtccgtacaccgaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccattcctgccccccgcactcccggagccacccggaatcctgggtccccagccccccgatgtgggctcctcggaccctctgagcatggtgggaccttcccagggccgaagccccagctacgcttcctga

SEQ ID NO: 7 - DNA, 2355 bp, Artificial Sequence
  Synthetic DNA
gcagactccagtcctctcctgcaattcgggggccaagtccggcagcggtacctctacacagatgatgcccagcgtacagaagcccacctggagatcagggaggatgggacggtggggggcgctgctgaccagagccccgaaagtctcctgcagctgaaagccttgaagccgggagttattcaaatcttgggagtccgtacaccgaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccattcctgccccccgcactcccggagccacccggaatcctgggtccccagccccccgatgtgggctcctcggaccctctgagcatggtgggaccttcccagggccgaagccccagctacgcttccggtggtggtggttctggtggtggtggttctggtggtggtggttctagaggtgttttcagaagagacgctcacaagtctgaagttgctcacagattcaaggacttgggtgaagaaaacttcaaggctttggttttgatcgctttcgctcaatacttgcaacaatgtccattcgaagaccacgttaagttggttaacgaagttactgaatttgctaagacttgtgttgctgacgaatctgctgaaaactgtgacaagtctttgcacactttgttcggtgacaagttgtgtactgttgctactttgagagaaacttacggtgaaatggctgactgttgtgctaagcaagaaccagaaagaaacgaatgtttcttgcaacacaaggacgacaacccaaacttgccaagattggttagaccagaagttgacgttatgtgtactgctttccacgacaacgaagaaactttcttgaagaagtacttgtacgaaatcgctagaagacacccatacttctacgctccagaattgttgttcttcgctaagagatacaaggctgctttcactgaatgttgtcaagctgctgacaaggctgcttgtttgttgccaaagttggacgaattgagagacgaaggtaaggcttcttctgctaagcaaagattgaagtgtgcttctttgcaaaagttcggtgaaagagctttcaaggcttgggctgttgctagattgtctcaaagattcccaaaggctgaatttgctgaagtttctaagttggttactgacttgactaaggttcacactgaatgttgtcacggtgacttgttggaatgtgctgacgacagagctgacttggctaagtacatctgtgaaaaccaagactctatctcttctaagttgaaggaatgttgtgaaaagccattgttggaaaagtctcactgtatcgctgaagttgaaaacgacgaaatgccagctgacttgccatctttggctgctgacttcgttgaatctaaggacgtttgtaagaactacgctgaagctaaggacgttttcttgggtatgttcttgtacgaatacgctagaagacacccagactactctgttgttttgttgttgagattggctaagacttacgaaactactttggaaaagtgttgtgctgctgctgacccacacgaatgttacgctaaggttttcgacgaatttaagccattggttgaagaaccacaaaacttgatcaagcaaaactgtgaattgttcgaacaattgggtgaatacaagttccaaaacgctttgttggttagatacactaagaaggttccacaagtttctactccaactttggttgaagtttctagaaacttgggtaaggttggttctaagtgttgtaagcacccagaagctaagagaatgccatgtgctgaagactacttgtctgttgttttgaaccaattgtgtgttttgcacgaaaagactccagtttctgacagagttactaagtgttgtactgaatctttggttaacagaagaccatgtttctctgctttggaagttgacgaaacttacgttccaaaggaatttaacgctgaaactttcactttccacgctgacatctgtactttgtctgaaaaggaaagacaaatcaagaagcaaactgctttggttgaattggttaagcacaagccaaaggctactaaggaacaattgaaggctgttatggacgacttcgctgctttcgttgaaaagtgttgtaaggctgacgacaaggaaacttgtttcgctgaagaaggtaagaagttggttgctgcttctcaagctgctttgggtttgtga

SEQ ID NO: 8 - DNA, 2355 bp, Artificial Sequence
  Synthetic DNA
agaggtgttttcagaagagacgctcacaagtctgaagttgctcacagattcaaggacttgggtgaagaaaacttcaaggctttggttttgatcgctttcgctcaatacttgcaacaatgtccattcgaagaccacgttaagttggttaacgaagttactgaatttgctaagacttgtgttgctgacgaatctgctgaaaactgtgacaagtctttgcacactttgttcggtgacaagttgtgtactgttgctactttgagagaaacttacggtgaaatggctgactgttgtgctaagcaagaaccagaaagaaacgaatgtttcttgcaacacaaggacgacaacccaaacttgccaagattggttagaccagaagttgacgttatgtgtactgctttccacgacaacgaagaaactttcttgaagaagtacttgtacgaaatcgctagaagacacccatacttctacgctccagaattgttgttcttcgctaagagatacaaggctgctttcactgaatgttgtcaagctgctgacaaggctgcttgtttgttgccaaagttggacgaattgagagacgaaggtaaggcttcttctgctaagcaaagattgaagtgtgcttctttgcaaaagttcggtgaaagagctttcaaggcttgggctgttgctagattgtctcaaagattcccaaaggctgaatttgctgaagtttctaagttggttactgacttgactaaggttcacactgaatgttgtcacggtgacttgttggaatgtgctgacgacagagctgacttggctaagtacatctgtgaaaaccaagactctatctcttctaagttgaaggaatgttgtgaaaagccattgttggaaaagtctcactgtatcgctgaagttgaaaacgacgaaatgccagctgacttgccatctttggctgctgacttcgttgaatctaaggacgtttgtaagaactacgctgaagctaaggacgttttcttgggtatgttcttgtacgaatacgctagaagacacccagactactctgttgttttgttgttgagattggctaagacttacgaaactactttggaaaagtgttgtgctgctgctgacccacacgaatgttacgctaaggttttcgacgaatttaagccattggttgaagaaccacaaaacttgatcaagcaaaactgtgaattgttcgaacaattgggtgaatacaagttccaaaacgctttgttggttagatacactaagaaggttccacaagtttctactccaactttggttgaagtttctagaaacttgggtaaggttggttctaagtgttgtaagcacccagaagctaagagaatgccatgtgctgaagactacttgtctgttgttttgaaccaattgtgtgttttgcacgaaaagactccagtttctgacagagttactaagtgttgtactgaatctttggttaacagaagaccatgtttctctgctttggaagttgacgaaacttacgttccaaaggaatttaacgctgaaactttcactttccacgctgacatctgtactttgtctgaaaaggaaagacaaatcaagaagcaaactgctttggttgaattggttaagcacaagccaaaggctactaaggaacaattgaaggctgttatggacgacttcgctgctttcgttgaaaagtgttgtaaggctgacgacaaggaaacttgtttcgctgaagaaggtaagaagttggttgctgcttctcaagctgctttgggtttgggtggtggtggttctggtggtggtggttctggtggtggtggttctgcagactccagtcctctcctgcaattcgggggccaagtccggcagcggtacctctacacagatgatgcccagcgtacagaagcccacctggagatcagggaggatgggacggtggggggcgctgctgaccagagccccgaaagtctcctgcagctgaaagccttgaagccgggagttattcaaatcttgggagtccgtacaccgaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccattcctgccccccgcactcccggagccacccggaatcctgggtccccagccccccgatgtgggctcctcggaccctctgagcatggtgggaccttcccagggccgaagccccagctacgcttcctga

SEQ ID NO: 9 - DNA, 1197 bp, Artificial Sequence
  Synthetic DNA
gcagactccagtcctctcctgcaattcgggggccaagtccggcagcggtacctctacacagatgatgcccagcgtacagaagcccacctggagatcagggaggatgggacggtggggggcgctgctgaccagagccccgaaagtctcctgcagctgaaagccttgaagccgggagttattcaaatcttgggagtccgtacaccgaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccattcctgccccccgcactcccggagccacccggaatcctgggtccccagccccccgatgtgggctcctcggaccctctgagcatggtgggaccttcccagggccgaagccccagctacgcttccggtggtggtggttctggtggtggtggttctggtggtggtggttctgttgaagaaccacaaaacttgatcaagcaaaactgtgaattgttcgaacaattgggtgaatacaagttccaaaacgctttgttggttagatacactaagaaggttccacaagtttctactccaactttggttgaagtttctagaaacttgggtaaggttggttctaagtgttgtaagcacccagaagctaagagaatgccatgtgctgaagactacttgtctgttgttttgaaccaattgtgtgttttgcacgaaaagactccagtttctgacagagttactaagtgttgtactgaatctttggttaacagaagaccatgtttctctgctttggaagttgacgaaacttacgttccaaaggaattcaacgctgaaactttcactttccacgctgacatctgtactttgtctgaaaaggaaagacaaatcaagaagcaaactgctttggttgaattggttaagcacaagccaaaggctactaaggaacaattgaaggctgttatggacgacttcgctgctttcgttgaaaagtgttgtaaggctgacgacaaggaaacttgtttcgctgaagaaggtaagaagttggttgctgcttctcaagctgctttgggtttgtaa

SEQ ID NO: 10 - DNA, 1197 bp, Artificial Sequence
  Synthetic DNA
gttgaagaaccacaaaacttgatcaagcaaaactgtgaattgttcgaacaattgggtgaatacaagttccaaaacgctttgttggttagatacactaagaaggttccacaagtttctactccaactttggttgaagtttctagaaacttgggtaaggttggttctaagtgttgtaagcacccagaagctaagagaatgccatgtgctgaagactacttgtctgttgttttgaaccaattgtgtgttttgcacgaaaagactccagtttctgacagagttactaagtgttgtactgaatctttggttaacagaagaccatgtttctctgctttggaagttgacgaaacttacgttccaaaggaattcaacgctgaaactttcactttccacgctgacatctgtactttgtctgaaaaggaaagacaaatcaagaagcaaactgctttggttgaattggttaagcacaagccaaaggctactaaggaacaattgaaggctgttatggacgacttcgctgctttcgttgaaaagtgttgtaaggctgacgacaaggaaacttgtttcgctgaagaaggtaagaagttggttgctgcttctcaagctgctttgggtttgggtggtggtggttctggtggtggtggttctggtggtggtggttctgcagactccagtcctctcctgcaattcgggggccaagtccggcagcggtacctctacacagatgatgcccagcgtacagaagcccacctggagatcagggaggatgggacggtggggggcgctgctgaccagagccccgaaagtctcctgcagctgaaagccttgaagccgggagttattcaaatcttgggagtccgtacaccgaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccattcctgccccccgcactcccggagccacccggaatcctgggtccccagccccccgatgtgggctcctcggaccctctgagcatggtgggaccttcccagggccgaagccccagctacgcttcctaa

SEQ ID NO: 11 - protein, 178 aa, Artificial Sequence
  Protein translated from synthetic DNA
MRDSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVCLSSAKQRQLYKNRGFLPLPGLPPALPEPPGILAPQPPDVGSSDPLSMVGPSQGRSPSYAS

SEQ ID NO: 12 - protein, 189 aa, Artificial Sequence
  Protein translated from synthetic DNA
MRDSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVCLSSAKQRQLYKNRGFLPLCHFLPMLPMVPEEPEDLRGHLESDMFSSPPDVGSSDPLSMVGPSQGRSPSYAS

SEQ ID NO: 13 - protein, 221 aa, Artificial Sequence
  Protein translated from synthetic DNA
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLCHFLPMLPMVPEEPEDLRGHLESDMFSSPLETDSMDPFGLVTGLEAVRSPSFEK

SEQ ID NO: 14 - protein, 210 aa, Artificial Sequence
  Protein translated from synthetic DNA
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPLETDSMDPFGLVTGLEAVRSPSFEK

(19) United States
(12) Patent Application Publication
(10) Pub. No.: US 2022/0111007 A1
(43) Pub. Date: Apr. 14, 2022

(54) Application of FGF21 in preparation of medicine for treating colorectal cancer

(71) Applicant: Jiangnan University, Wuxi (CN)

(72) Inventors: Shenglong Zhu, Wuxi (CN); Yongquan Chen, Wuxi (CN); Zhen Wang, Wuxi (CN)

(21) Appl. No.: 17/559,050

(22) Filed: Dec. 22, 2021

Related U.S. Application Data
(63) Continuation of application No. PCT/CN2020/136762, filed on Dec. 16, 2020.

(30) Foreign Application Priority Data
May 12, 2020 (CN) 2020103960431

(51) Int. Cl. A61K 38/18; A61P 35/00

(52) U.S. Cl. CPC A61K 38/1825; A61P 35/00

(57) ABSTRACT

The present invention discloses application of FGF21 in the preparation of medicines for treating colorectal cancer, and belongs to the technical field of medicine. The FGF21 protein provided by the present invention is a secreted protein composed of about 210 amino acids. The present invention found that FGF21 has the effect of treating colorectal tumors for the first time. It is proved by cytology and animal experiments that FGF21 can inhibit the proliferation, invasion and migration of colorectal tumor cell lines; can significantly reduce the size of tumor lesions in mouse models. The medicines prepared from FGF21 as the active ingredient for treating colorectal cancer has good safety and long duration of drug action, while it protects organs from damage and has the effect of improving lipid metabolism disorders.

### TECHNICAL FIELD

The present invention relates to the application of FGF21 in the preparation of medicines for treating colorectal cancer, in particular to the application of human FGF21 in the preparation of medicines for treating colorectal cancer, and belongs to the technical field of medicine.

### BACKGROUND

With 1023152 new cases each year around the world, colorectal cancer ranks third in the incidence of common malignant tumors, accounting for 10.38% of the total incidence of malignant tumors.

Currently, the treatment of colorectal cancer is mainly based on radical surgery, with chemotherapy, radiotherapy, traditional Chinese medicine treatment and biological treatment as its auxiliary treatment means. Approximately two-thirds of patients diagnosed for the first time underwent radical surgery, and more than half of the patients experienced recurrence or distant metastasis. For advanced cancer or cancer with extensive metastasis, radical surgery cannot be performed, and comprehensive medical treatment often becomes the main treatment means. Among operable patients, the recurrence rate is still higher after colorectal cancer surgery. The commonly used therapeutic drugs are usually broad-spectrum chemical drugs with many side effects. Therefore, the development of a safe drug for treating colorectal cancer that has the effect of inhibiting tumor growth and recurrence as well as the organ protection effect without related obvious side effects is the ideal goal of current research and development.

In recent years, a large number of clinical investigations have shown that the occurrence and development of colorectal cancer are closely related to metabolic abnormalities. A large number of studies have found that metabolic pathways including glycolysis, tricarboxylic acid cycle, fatty acid metabolism, glutamine metabolism and the like have undergone reprogramming changes in tumor cells, and tumor cells can provide fatty acids, nucleotides and the like required for proliferation by coordinating various metabolic pathways, such that the synthesis process of biological macromolecules is more suitable for the rapid proliferation of tumor cells. In particular, it should be pointed out that metabolic abnormalities are not only the result of tumor occurrence and development, but may also be directly involved in the initiation process of tumors. Currently, the development of anti-tumor drugs from the perspective of metabolism has been successfully applied in many tumors. For example, recent studies have found that some genes encoding metabolic enzymes are oncogenes themselves, for example, the mutation of fumarate carboxylase is related to the occurrence and development of renal cancer; the mutation of succinate carboxylase is related to the occurrence and development of pheochromocytoma; the mutation of isocitrate dehydrogenase produces metabolites of small molecules 2HG, which inhibits the expression of HNF4α and thus induces cholangiocarcinoma and the like. Based on the above analysis, it is indicated that targeting tumor cell metabolism may be one of the effective ways to kill tumor cells. However, the drugs currently developed are all small molecule inhibitors with poor targeting and more side effects, while bioprotein drugs are absorbed quickly and have strong targeting. Therefore, the development of new targeted bioprotein preparations for colorectal cancer is currently a hot spot in tumor research.

### SUMMARY

The present invention has discovered through a large number of studies that human fibroblast growth factor 21, which is an important member involved in multiple links of the glycolipid metabolism network, can significantly inhibit the occurrence, development and metastasis of colorectal cancer. Based on this, the present invention discloses a new application of FGF21 protein in the preparation of medicines for preventing, relieving and/or treating colorectal cancer.

The present invention discloses the application of FGF21 protein in the preparation of medicines for preventing, relieving and/or treating colorectal cancer.

In one implementation, the amino acid sequence of the FGF21 protein is as set forth in any one of SEQ ID NO. 1-5 or SEQ ID NO. 11-14.

In one implementation, the effective dose of the FGF21 protein is 0.1-10 mg/kg.

In one implementation, the administration route of the medicine includes oral, intraperitoneal injection, subcutaneous injection, intravenous injection or intramuscular injection.

The present invention discloses a medicine for treating colorectal cancer, and the active ingredient of the medicine includes FGF21 protein.

In one implementation, the amino acid sequence of the FGF21 protein is as set forth in any one of SEQ ID NO. 1-5 or SEQ ID NO. 11-14.

In one implementation, the medicine further includes a pharmaceutically acceptable carrier or accessory.

In one implementation, the administration route of the medicine includes oral, intraperitoneal injection, subcutaneous injection, intravenous injection or intramuscular injection.

The present invention discloses the application of FGF21 gene as a medicine target in the screen of medicines for treating colorectal cancer.

In one implementation, the nucleotide sequence of the FGF21 gene is as set forth in any one of SEQ ID NO. 6-10.

In one implementation, the application is for screening small molecule activators of FGF21 protein.

The present invention discloses the application of FGF21 protein in the preparation of inhibitors of sugar metabolism and/or glutamine metabolism in tumor.

In one implementation, the application is for preparing the inhibitors of one or more of HK2, PKM2, PFKP, PD, and GPD that are key proteins for inhibiting the sugar metabolism in tumor.

In one implementation, the amino acid sequence of the FGF21 protein is as set forth in any one of SEQ ID NO. 1-5 or SEQ ID NO. 11-14.

In one implementation, the application is for preparing an inhibitor of GLS, a protein related to glutamine metabolism.

The present invention discloses that the application of FGF21 protein in the preparation of medicines for preventing, relieving and/or treating one or more diseases of prostate cancer, esophageal cancer, colorectal adenocarcinoma, cervical cancer, endometrial cancer, ovarian cancer, pancreatic cancer, gallbladder cancer, liver cancer, clear cell kidney cancer, melanoma, and multiple myeloma.

In one implementation, the amino acid sequence of the FGF21 protein is as set forth in any one of SEQ ID NO. 1-5 or SEQ ID NO. 11-14.

The present invention has the following beneficial effects compared with the prior art:

(1) the present invention has discovered new functions of FGF21 gene, including the ability to significantly change tumor metabolism, especially significantly inhibit the key proteins HK2, PKM2 and GLS in the process of sugar metabolism and glutamine metabolism in tumor, and inhibit the proliferation, invasion and migration of colorectal tumor cell lines; and the ability to significantly reduce the size of tumor lesions in a mouse model, so that it can be used as an active ingredient in various dosage forms of drugs, and is of great significance for the treatment of colorectal cancer.

(2) the present invention can relieve the occurrence and development of colorectal cancer by injecting FGF21 protein, and inhibit the generation and metastasis of colon cancer.

### BRIEF DESCRIPTION OF FIGURES

FIG. 1 shows the effect of FGF21 on the growth rate of human colorectal cancer xenografts, indicating the change in tumor volume. and B is the change in tumor quality.

FIG. 2 shows the effect of FGF21 on the growth rate of human colorectal cancer xenografts, indicating the change in tumor quality.

FIG. 3 shows the effect of FGF21 AOM/DSS-induced colorectal cancer in mice, indicating the number of large intestine tumors.

FIG. 4 shows the effect of FGF21 AOM/DSS-induced colorectal cancer in mice, indicating the morphology of the large intestine.

FIG. 5 shows the effect of FGF21 on the key protein of tumor metabolism, indicating the change in the key protein of sugar metabolism pathway in tumor.

FIG. 6 shows the effect of FGF21 on the key protein of tumor metabolism, indicating the change in the key protein of glutamine metabolism in tumor.

### EMBODIMENT

Experimental animals and feeding: Nude mice and C57BL/6 mice were purchased from Shanghai SLAC. They were raised in the Animal Center in Wuxi School of Medicine, Jiangnan University under a 12-hour light/dark cycle at a temperature of 20±2° C.

Cell culture: Colorectal cancer cell line SW620 was provided by the Institute of Biochemistry and Cell Biology, Chinese Academy of Sciences;

RPM11640, DMEM, and 0.05% Trypsin were purchased from Boster Company; fetal bovine serum was purchased from Sijiqing Company. Azoxymethane was purchased from Sigma Company (batch number 098k1488), Dextran Sulfate Sodium MP Biomedicals (batch number 0216011050); other drugs were domestically produced analytically pure. Colorectal cancer cell lines were grown adherently in RPMI 1640 culture medium containing 10% fetal bovine serum, cultured in a humidified incubator at 37° C. and 5% CO₂, and passaged every other day.

Luminescence kit was purchased from: Millipore Company.

### Embodiment 1: Application of FGF21 in the Preparation of Medicines

FGF21 protein was prepared by culturing microbial cells, the specific method includes:

(1) Construction of Recombinant Plasmid

According to the codon preference of E. coli, a gene encoding the FGF21 protein with the amino acid sequence as set forth in SEQ ID NO. 1 was designed and sent to the biological company for synthesis, while two restriction sites, NdeI and BamHI, were designed at both ends of the gene.

The synthesized gene fragment was connected to the vector pET30a(+) through NdeI and BamHI to construct the recombinant plasmid pET30a-mFGF21.

(2) Expression of Target Protein

The recombinant plasmid pET30a-mFGF21 containing the correct sequence was transformed into the expression strain Rosseta (DE3) (TransGen Biotech Co., Ltd, Beijing, catalog number: CD801). The transformed single colonies were respectively inoculated into 20 mL of LB medium containing Kan (50 μg/mL), cultured at 37° C. for 8 h, inoculated into another 20 mL of LB medium containing Kan (50 μg/mL) at a volume ratio of 1:100, and cultured at 37° C. When A600 was around 0.35, IPTG was added to achieve a final concentration of 0.25 mmol/L for induction with an induction temperature of 30° C. After 5 h, the bacterial cells were harvested, resuspended with Lysis buffer (20 mmol/L Tris, 150 mmol/L NaCl, pH 8.0), broken and centrifuged, and the supernatant and the precipitate were respectively taken for 12 wt % SDS-PAGE electrophoresis analysis.

(3) Purification of Protein

The bacterial cells were added with a certain concentration of lysozyme (1 mg/mL), placed on ice for 30 min, and broken by ultrasonic cell crusher (working 1 s with interval of 1 s, 4 min/time, 3 cycles in total). After the bacterial cells were completely broken, the Quix Stand pretreatment system (750 kD hollow fiber ultrafiltration column) was used to treat the cell crushing liquid to enrich the inclusion bodies, and the liquid at the permeate end of the membrane was discarded. When the total volume was about 60 mL, 100 mL wash buffer (20 mmol/L Tris, 2 mol/L Urea, 150 mmol/L NaCl, pH 8.0) were added to wash the inclusion bodies. When the volume of the solution is 50 mL, 100 mL washing solution were added, and the above experiment procedure was repeated for 4 times.

After washing, when the volume of the solution was 50 mL, the permeate end was closed, 150 mL of denaturation solution (20 mmol/L Tris, 10 mol/L Urea, 150 mmol/L NaCl, pH 8.0) were added to the washed inclusion bodies, and the denaturation was cycled for 2 h. The permeate end was opened, and the collected liquid at the permeate end of the membrane was mFGF21 denaturation solution. The denatured mFGF21 was concentrated to a volume of 80 mL with a 5 KD hollow fiber column for renaturation, and the container containing the renaturation solution (20 mmol/L Tris, 50 mmol/L NaCl, pH 8.0) was connected to the reservoir of the hollow fiber column with a rubber tube. After the reservoir was sealed and the liquid flowed out from the permeate end, the renaturation solution was dripped into the denaturation solution at a certain rate due to the negative pressure generated in the reservoir, so that the renaturation was performed slowly and uniformly. When the added volume of the renaturation solution was 6 times that of the denaturation solution, the renaturation was completed, the centrifugation was carried out at 8000 rpm/min, 4° C. for 20 min, and the supernatant was collected. The renaturation supernatant was passed through the AKTA purifier 100 system, completely bound with the Capto Q column (installed in the XK16/20 empty column with a column height of 10 cm and a flow rate of 300 cm/h) equilibrated with 5 column volumes of IEX buffer A (20 mmol/L Tris, 10 mmol/L NaCl, pH 8.0), and then rinsed with 3-4 column volumes of IEX buffer A. When the UV curve reached a stable baseline, a mixture of IEX buffer A and IEX buffer B (20 mmol/L Tris, 1 mol/L NaCl, PH 8.0) was used for elution. 15 wt % and 100 wt % IEX buffer B were used to rinse the impure proteins, and 18.5 wt %-19 wt % IEX buffer B were used to elute the target protein. Each elution peak was collected, and 15 wt % SDS PAGE electrophoresis analysis was carried out. The purified FGF21 protein was obtained.

The FGF21 protein prepared by microbial cells was used together with pharmaceutically acceptable carriers or accessories to prepare medicines, and the pharmaceutically acceptable carriers or accessories may be excipients, adjuvants and/or diluents.

### Embodiment 2: Effect of FGF21 on the Growth Rate of Human Colorectal Cancer Xenografts

FGF21 protein with the amino acid sequence as set forth in SEQ ID NO. 1 was prepared according to the method of Embodiment 1.

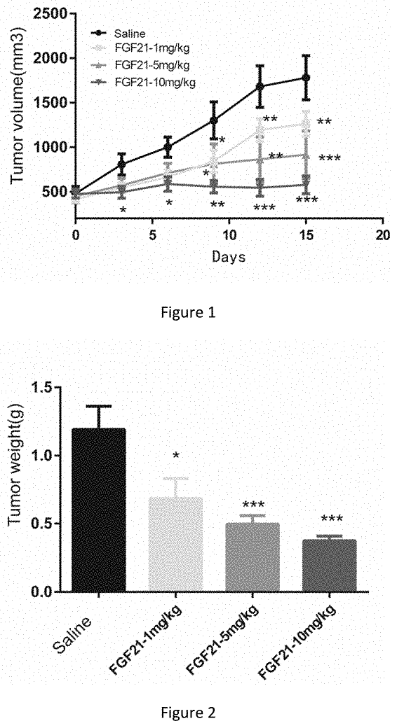

SW620 cells, human colon cancer cells, were subcutaneously inoculated into the 6-week-old male nude mice at 1×10⁶ cells/mouse. When the tumor grew to 200 mm³, they were randomly divided into four groups: (1) normal saline group: injection with the same volume of normal saline as the experimental group; (2) low-dose group: injection with 1 mg/kg FGF21; (3) medium-dose group: injection with 5 mg/kg FGF21; and (4) high-dose group: injection with 10 mg/kg FGF21. The mice were injected once a day for 15 consecutive days. The tumor volume was monitored every day, the mice were sacrificed three weeks later, and the tumor weight was weighed. The results showed that the three different doses of FGF21 could inhibit the xenograft volume and the final tumor weight in a dose-dependent manner. After injecting with high-dose of FGF21 (10 mg/kg), it can completely inhibit tumor growth and maintain the tumor volume at 500 mm³, the final volume was only 30% of the control group, Similarly, the tumor weight had also reduced by about 70%. Injection with 1 mg/kg and 5 mg/kg can reduce tumor weight by 30% and 50%, respectively (as shown in FIG. 1 and FIG. 2).

### Embodiment 3: Effect of FGF21 on AOM/DSS-Induced Colorectal Cancer in Mice

FGF21 protein with the amino acid sequence as set forth in SEQ ID NO. 1 was prepared according to the method of Embodiment 1.

C57 mice were injected with 10 mg/kg of azoxymethane in the first week and had a free access cycle to 2.5% dextran sulfate sodium at the same time. Each cycle consisted of three weeks (21 days). The mice drank water containing dextran sulfate sodium in the first week and ordinary water in the next two weeks for a total of three cycles with 3 weeks per cycle. At the sixth week, they were divided into normal saline group and FGF21 injection group. The FGF21 injection group was injected at a dose of 10 mg/kg. The normal saline group was injected with the same amount of normal saline once a day for 4 consecutive weeks.

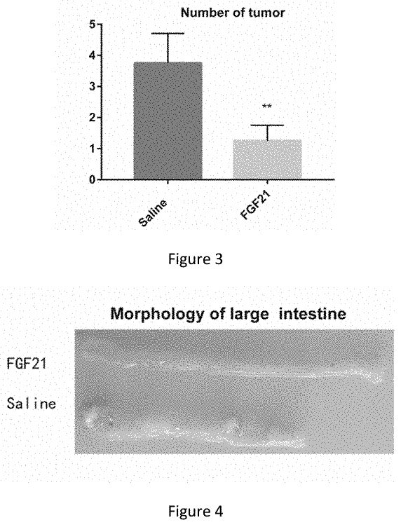

The results were shown in FIG. 3. After injection of FGF21, the average number of tumor lesions can be reduced from 4 to 1. FIG. 4 showed typical pictures of the large intestine for the FGF21 treatment group and the control group, which can intuitively show the therapeutic effect of FGF21 including reducing the number of tumors, improving the problem of shortened colon length, and increasing the length of the colon by 10-20 mm compared with the control group with an average increase of 18 mm.

### Embodiment 4: Effect of FGF21 on Key Proteins in Tumor Metabolism

FGF21 protein with the amino acid sequence as set forth in SEQ ID NO. 1 was prepared according to the method of Embodiment 1.

The level of tumor metabolism-related proteins in the tumor tissues of Embodiment 3 was detected by Western Blot (protein immunoblotting):

a) Formulation of Solution:

(1) 10% (w/v) sodium dodecyl sulfate (SDS) solution: 0.1 g of SDS and 1 ml H₂O deionized water was used for preparation, and the solution was stored at room temperature.

(2) separation gel buffer: 1.5 mmol/L Tris-HCL (pH 8.8): 18.15 g Tris was dissolved in 80 ml of water, adjusted to pH 8.8 with HCl, and diluted with water to a final volume of 100 ml.

(3) concentrated gel buffer: 0.5 mmol/L Tris-HCL (pH 6.8): 6.05 g Tris was dissolved in 80 ml of water, adjusted to pH 6.8 with HCl, and diluted with water to a final volume of 100 ml.

(4) SDS-PAGE loading buffer: 8 ml of 0.5 mol/L Tris buffer at pH 6.8, 6.4 ml glycerol, 12.8 ml of 10% SDS, 3.2 ml of mercaptoethanol, 1.6 ml of 0.05% bromophenol blue, and 32 ml of H₂O were mixed well for later use.

(5) Tris-glycine electrophoresis buffer: 303 g Tris, 188 g glycine and 10 g SDS were dissolved in distilled water to 1000 ml, and diluted 10 times before use.

(6) membrane transfer buffer: 14.4 g glycine and 6.04 g Tris were weighed, added with 200 ml methanol, and added with water to a total volume of 1 L.

(7) Tris buffered salt solution (TBS): 20 mmol/L Tris/HCl (pH 7.5) and 500 mmol/L NaCl.

b) The specific steps were as follows: part of the transplanted tumor cells in Embodiment 2 was taken and washed 3 times with PBS, the lysate was added, the mixture was boiled directly for 5 min, cooled on ice, and centrifuged at 12000 rpm for 2 min, and the supernatant taken and stored at −20° C. for later use.

(A) Determination of Protein Concentration

BCA method was used to determine protein concentration: 0.5 mg/mL standard protein gradient was added to the well of the plate, and PBS was added to make up to 20 μL; an appropriate volume (3 μL) of protein sample was added to the well of the plate, and PBS was added to make up to 20 μL; 200 μL BCA working solution (formulated immediately before use) was added to each well and incubated at 37° C. for 30 min; the absorbance at 562 nm was measured, and the protein concentration was calculated from the standard curve and sample volume.

(B) SDS-PAGE Gel Electrophoresis

(1) Two cleaned glass plates are aligned and put into a clamp for clamping, and then vertically clamped on a shelf for glue pouring.

(2) 10% separation gel was prepared by adding TEMED and immediately and uniformly shaking. After that, the glue pouring was carried out and then the ethanol was used to seal. The ethanol on the upper layer of the gel was poured out and dried with absorbent paper when the gel was fully solidified.

(3) 5% concentrated gel was prepared by adding TEMED and immediately and uniformly shaking. After that, the glue pouring was carried out. The remaining space was filled with concentrated gel and then the comb was inserted into the concentrated gel. After the concentrated gel was solidified, it was gently pulled straight up.

(4) It was added into the electrophoresis tank and enough electrophoresis solution was added for sample loading. After measuring the protein content of the protein sample, 5×SDS loading buffer was added to 1× final concentration, the mixture was boiled in boiling water for 3 min and mixed well for sample loading with the total protein amount of 35 μg.

(5) Electrophoresis was carried out at constant pressure of 80 V to run the gel, when the sample entered the lower layer gel, electrophoresis was carried out at constant pressure of 120 V until bromophenol blue reached the bottom of the gel, and then membrane transfer was carried out.

(C) Membrane Transfer:

(1) Gel cutting: The glass plates were pried off, the concentrated gel was scraped off after the small glass plates were removed, and gel cutting was carried out according to the molecular weight of protein and experimental needs with Marker as the control.

(2) Membrane preparation: A PVDF membrane and filter paper were cut, and the cut PVDF was placed in a 80% methanol solution for activation 30 s.

(3) Membrane loading: A clamp used in membrane transfer was released to keep a black side (negative electrode) level. A sponge pad was placed on the black side, the transfer buffer was added to soak the sponge pad, the soaked filter paper was placed on the pad, and then the gel, a nitrocellulose membrane, the filter paper and the sponge pad were sequentially stacked in order. Finally, a white plate (positive electrode) was placed and loaded into a membrane transfer tank.

(4) Membrane transfer: The clamp was placed into the membrane transfer tank to make the black side of the clamp face the black side of the tank and make the white side of the clamp face the red side of the tank. Membrane transfer was carried out at 4° C. and constant current of 400 mA.

(5) The membrane was removed after membrane transfer, and a corner was marked.

(D) Immune Response

(1) Blocking: the membrane was rinsed 3 times in TBST for 5 min each time. After rinsing, the nitrocellulose membrane was added into 5% skimmed milk powder and shaken for 1 h at room temperature.

(2) Addition of primary antibody: the blocked nitrocellulose membrane was added into a washing tank containing TBST buffer and rinsed 3 times on a shaker for 5 min each time. It was added into a plate with primary antibody and incubated overnight at 4° C.

(3) Addition of secondary antibody: the primary antibody was recovered, the nitrocellulose membrane in a washing tank containing TBST buffer solution was rinsed 3 times on a shaker for 5 min each time at room temperature, and then the rinsed nitrocellulose membrane was added into a plate with secondary antibody and incubated for 45 min at room temperature in the dark. After the incubation, the nitrocellulose membrane was washed 3 times in a washing tank containing TBST for 5 min each time.

(E) Chemiluminescence

(1) Reagents A and B were mixed in a small centrifuge tube according to instructions of a luminescence kit and then added onto the nitrocellulose membrane, and a chemiluminescence imager was used for color developing.

Tumor cells maintain their energy requirements for abnormal proliferation and metastasis mainly through changes in metabolism, while changes in metabolic rate are mainly achieved by regulating the expression levels of key genes in the metabolic process. Among them, changes in glycolysis metabolism and glutamine metabolism are particularly important.

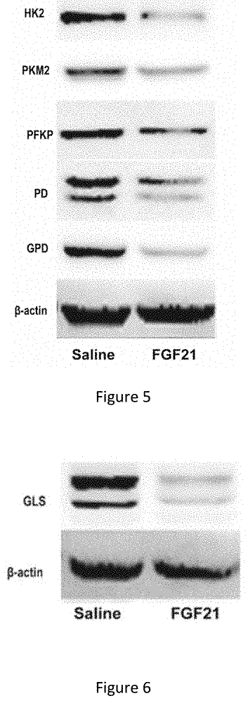

It can be seen from FIG. 5 and FIG. 6 that FGF21 injection can significantly inhibit HK2 (hexokinase 2), PKM2 (pyruvate kinase), PFKP (phosphofructokinase), PD (pyruvate dehydrogenase), and GPD (glucose-6-phosphate dehydrogenase) (FIG. 5) that are key proteins for sugar metabolism in tumor, and GLS (glutaminase) (FIG. 6) associated with glutamine metabolism. These proteins are highly expressed in the abnormal proliferation of tumors. A variety of drugs targeting to inhibit these proteins can significantly improve colorectal cancer. The inhibition of the expression of these key proteins by FGF21 further illustrates that FGF21 has a therapeutic effect on colorectal cancer and the anti-tumor effect of FGF21 on colorectal cancer cells is achieved mainly through the inhibition of glycolysis and glutamine metabolism.

The inventor also tried to study the effect of the fusion protein of FGF21 protein as set forth in SEQ ID NO. 2-5 in the treatment of colorectal cancer using the above method. The results show that the FGF21 fusion protein as set forth in any one of SEQ ID NO. 2-5 and/or SEQ ID NO. 11-14 has comparable effects to the FGF21 protein as set forth in SEQ ID NO. 1.

Although the present invention has been disclosed as above in preferred embodiments, it is not intended to limit the present invention. Those skilled in the art can make various changes and modifications without departing from the spirit and scope of the present invention. Therefore, the protection scope of the present invention should be defined by the claims.

What is claimed is:

1. A method of preparing a medicine for preventing, relieving and/or treating colorectal cancer, wherein the medicine comprises the FGF21 protein, the amino acid sequence is as set forth in any one of SEQ ID NO. 1-5 or SEQ ID NO. 11-14.

2. The method according to claim 1, wherein the effective dose of the FGF21 protein is 0.1-10 mg/kg.

3. The method according to claim 1, wherein the administration route of the medicine comprises oral, intraperitoneal injection, subcutaneous injection, intravenous injection or intramuscular injection.

4. A method of preparing a medicine targeting in the screen of medicines for treating colorectal cancer, wherein the medicine comprises FGF21 gene.

5. The method according to claim 4, wherein the medicine is for screening the small molecule activators of FGF21 protein.

6. The method according to claim 4, wherein the nucleotide sequence of the FGF21 gene is as set forth in any one of SEQ ID NO. 6-10.

7. A method of preparing inhibitors of sugar metabolism and/or glutamine metabolism in tumor, wherein the inhibitors comprise FGF21 protein.

8. The method according to claim 7, wherein the effective dose of the FGF21 protein is 0.1-10 mg/kg.

9. The method according to claim 8, wherein the method is for preparing the inhibitors of one or more of hexokinase 2, pyruvate kinase, phosphofructokinase, pyruvate dehydrogenase, and glucose-6-phosphate dehydrogenase that are key proteins for inhibiting the sugar metabolism in tumor.

10. The method according to claim 9, wherein the method is for preparing an inhibitor of glutaminase.
